(19) United States
(12) Patent Application Publication
(10) Pub. No.: US 2022/0184847 A1
(43) Pub. Date: Jun. 16, 2022

(54) METHOD FOR PRODUCING A CERAMIC MULTILAYER BLANK

(71) Applicant: DENTSPLY SIRONA INC., YORK, PA (US)

(72) Inventors: Stefan FECHER, Johannesberg (DE); Lothar VÖLKL, Goldbach (DE); Daniel AMMON, Webster, NY (US)

(73) Assignee: DENTSPLY SIRONA INC., YORK, PA (US)

(21) Appl. No.: 17/549,481

(22) Filed: Dec. 13, 2021

Related U.S. Application Data
(60) Provisional application No. 63/125,456, filed on Dec. 15, 2020.

(51) Int. Cl. B28B 1/16; B28B 11/24; C04B 35/48; C04B 35/057; C04B 35/04; C04B 35/50; C04B 35/645

(52) U.S. Cl. CPC B28B 1/16; B28B 11/243; C04B 35/48; C04B 35/057; C04B 35/04; C04B 35/50; C04B 2235/9661; C04B 2235/3246; C04B 2235/3208; C04B 2235/3206; C04B 2235/3229; C04B 2235/6027; C04B 2235/75; C04B 35/645

(57) ABSTRACT

The present invention is related to a method for producing a ceramic multilayer blank comprising at least a first layer of a first ceramic material and at least a second layer of a second ceramic material, wherein the first layer and the second layer are made of ceramic materials of different compositions, which are filled in pourable condition layer-by-layer into a mold and thereafter they are pressed and then sintered, wherein the first layer is a pink colored layer, wherein the first ceramic material comprises 2 to 25 wt % erbium oxide.

### CROSS-REFERENCE TO RELATED APPLICATIONS

This Application claims the benefit of and priority to Provisional Application Ser. No. 63/125,456, filed Dec. 15, 2020, which is herein incorporated by reference for all purposes.

### FIELD OF THE INVENTION

The present invention relates to a method for producing a ceramic multilayer blank comprising at least a first layer of a first ceramic material and at least a second layer of a second ceramic material, wherein the first layer and the second layer are made of ceramic materials of different compositions, which are filled in pourable condition layer-by-layer into a mold and thereafter they are pressed and then sintered.

### BACKGROUND OF THE INVENTION

U.S. Pat. No. 8,936,848 B2 discloses a blank of zirconium dioxide that is used for the preparation of a tooth replacement and comprises a number of layers of different chemical compositions. The individual layers thereby have different percentages of yttrium oxide.

A body of zirconium dioxide exhibits a decrease or increase in chromaticity along a straight line in the L*a*b* color space.

A blank of zirconium dioxide for the preparation of dental objects in accordance with WO 20141062375 A1 has at least two material regions which have different proportions of tetragonal and cubic crystal phases, wherein in one of the regions the ratio is greater than 1 and in the other region the ratio is lower than 1.

EP 2 371 344 A1 relates to a ceramic body which is enriched with a stabilizing agent from the surface to a desired depth.

Zirconium dioxide is used as a ceramic material to produce dental restorations. A frame-work can be milled, for example, from a blank of zirconium dioxide and can then be sintered. In the following processing stages, a veneer is applied manually to the framework, wherein at least one incisor material is applied manually and fused. All of these process steps are time-consuming.

Even when many ceramic materials for dental restorations based on a zirconium dioxide system have already been investigated, it has been found very challenging up to the present to produce a specific ceramic base material, which is able to provide a pink color having a high similarity to human gingiva.

The provision of such a pink colored ceramic material and methods for producing them are more and mare required by the dental industry for dental restorations, such as for the production of a full or a partial denture, an implant supported superstructure, or an implant supported denture.

In view of the prior art, it was thus an object of the present invention to provide a method to produce a pink colored ceramic material for dental restoration applications wherein said pink colored ceramic material shall be as similar as possible to human gingiva.

### SUMMARY OF THE INVENTION

These objects and further objects which are not stated explicitly but are immediately derivable or discernible from the connections discussed herein by way of introduction are achieved by a method having all features of claim 1. Appropriate modifications to the inventive method are protected in dependent claims 2 to 18.

The present invention accordingly provides a method for producing a ceramic multilayer blank comprising at least a first layer of a first ceramic material and at least a second layer of a second ceramic material, wherein the first layer and the second layer are made of ceramic materials of different compositions, which are filled in pourable condition layer-by-layer into a mold and thereafter they are pressed and then sintered, characterized in that the first layer is a pink colored layer, wherein the first ceramic material comprises 2 to 25 wt %, preferably 4 to 17 wt %, and more preferably 5 to 12 wt %, erbium oxide.

It is thus possible in an unforeseeable manner to provide a method to produce a pink colored ceramic material for dental restoration applications wherein said pink colored ceramic material is similar to human gingiva.

### BRIEF DESCRIPTION OF THE FIGURES

For a more complete understanding of the present invention, reference is made to the following Detailed Description of the Invention considered in conjunction with the accompanying figures, in which:

FIG. 1 exhibits a schematic of an assembly and the process steps that can be carried out with it in accordance with the present invention.

FIG. 2 exhibits the assembly shown in FIG. 1b) in greater detail.

FIG. 3 exhibits a schematic of an alternative method in accordance with the present invention.

### DETAILED DESCRIPTION OF THE INVENTION

The expression “substantially free” means in the context of the present invention a concentration of less than 0.0005 weight percent, preferably less than 0.0003 weight percent, and more preferably less than 0.0001 weight percent.

In one embodiment, the ceramic multilayer blank additionally comprises at least a third layer, wherein said third layer is made of a third ceramic material, which has a different composition than the first layer, and which has a different or identical composition than the second layer; wherein after filling of the second layer of the second ceramic material in pourable condition, surface of the second layer is structured in such a way that the second layer when viewed across its surface differs from region to region in its height, and then as the third layer, a layer of a third ceramic material in pourable condition is filled into the mold; or

wherein after filling of the second layer of the second ceramic material in pourable condition, an intermediate layer of an intermediate ceramic material in pourable condition, which differs from the second layer, is filled into the mold; wherein the second ceramic material of the second layer is mixed with the third ceramic material of the third layer to form said intermediate layer of an intermediate ceramic material; and then the third layer of a third ceramic material in pourable condition is filled into the mold.

In an alternative embodiment thereto, the ceramic multilayer blank further comprises a plurality of additional layers, wherein each of said additional layers is made of a ceramic material, which has a different composition than the first layer, and which has a different or identical composition than the second layer;

wherein after filling of the second layer of the second ceramic material in pourable condition, surface of the second layer is structured in such a way that the second layer when viewed across its surface differs from region to region in its height, and then as the first of said additional layers, a layer of a ceramic material of said additional layers in pourable condition is filled into the mold; wherein after filling of the first of said additional layers of a ceramic material of said additional layers in pourable condition, surface of the first of said additional layers is structured in such a way that the first of said additional layers when viewed across its surface differs from region to region in its height, and then as the second of said additional layers, a layer of a ceramic material of said additional layers in pourable condition is filled into the mold;

wherein if the second of said additional layers is not the last additional layer, the aforementioned method step is repeated until all additional layers in pourable condition are filled into the mold.

In one embodiment therein, the surface of the respective layer is structured in such a way that elevations and depressions are provided.

In another alternative embodiment, the ceramic multilayer blank further comprises a plurality of additional layers, wherein each of said additional layers is made of a ceramic material, which has a different composition than the first layer, and which has a different or identical composition than the second layer;

wherein after filling of the second layer of the second ceramic material in pourable condition, a first intermediate layer of a first intermediate ceramic material in pourable condition, which differs from the second layer, is filled into the mold; wherein the second ceramic material of the second layer is mixed with the ceramic material of the first of said additional layers to form said first inter-mediate layer of a first intermediate ceramic material; and then the first of said additional layers of a ceramic material in pourable condition is filled into the mold;

wherein after filling of the first of said additional layers of a ceramic material in pourable condition, a second of said additional layers of a ceramic material of said additional layers in pourable condition is filled into the mold; wherein the ceramic material of the first of said additional layers is mixed with the ceramic material of the second of said additional layers to form said second intermediate layer of a second intermediate ceramic material, and then the second of said additional layers is filled into the mold;

wherein if the second of said additional layers is not the last additional layer, the aforementioned method step is repeated until all additional layers in pourable condition are filled into the mold.

In one embodiment, the ceramic multilayer blank comprises two to five, preferably two to four, more preferably three, additional layers, wherein each of said additional layers is made of a ceramic material, which has a different composition than the first layer, and which has a different or identical composition than the second layer.

In one embodiment, the ceramic material of the second layer and/or the ceramic material of the third layer comprises at least one oxide of the elements Mn, Co, Fe, Tb, Pr and Er; and/or wherein the ceramic material of the first layer further comprises at least one oxide of the elements Mn, Co, Fe, Tb and Pr.

In one embodiment, the ceramic material of the second layer and/or the ceramic material of all additional layers comprises at least one oxide of the elements Mn, Co, Fe, Tb, Pr and Er; and/or wherein the ceramic material of the first layer further comprises at least one oxide of the elements Mn, Co, Fe, Tb and Pr.

In one embodiment, the ceramic material of the first layer and/or the ceramic material of the second layer and/or the ceramic material of the third layer and/or the ceramic material of all additional layers of the ceramic multilayer blank further comprises between 0.0005 and 0.02 wt %, preferably between 0.0005 and 0.01 wt %, and more preferably between 0.0005 and 0.05 wt % of an oxide of the element Mn; and/or

the ceramic material of the first layer and/or the ceramic material of the second layer and/or the ceramic material of the third layer and/or the ceramic material of all additional layers of the ceramic multilayer blank further comprises between 0.0005 and 0.1 wt %, preferably between 0.0005 and 0.07 wt %, and more preferably between 0.0005 and 0.05 wt % of an oxide of the element Co.

In one embodiment, the content of the at least one oxide of the element Mn in the respective ceramic material of the respective layer of the multilayer blank is continuously increasing from the second layer to the third layer; or from the second layer via the first of the additional layers continuously further to the last of the additional layers.

In one embodiment, the content of the at least one oxide of the element Mn is continuously increasing by totally 1 to 50 ppm, preferably by 1 to 30 ppm, and more preferably by 1 to 10 ppm; from the second layer to the third layer; or from the second layer to the last of the additional layers; wherein the ceramic multilayer blank is substantially, preferably completely, free of any oxide of the element Co.

In one embodiment, the content of the at least one oxide of the element Co in the respective ceramic material of the respective layer of the multilayer blank is continuously increasing from the second layer to the third layer; or from the second layer via the first of the additional layers continuously further to the last of the additional layers.

In one embodiment, the content of the at least one oxide of the element Co is continuously increasing by totally 1 to 100 ppm, preferably by 1 to 60 ppm, and more preferably by 1 to 30 ppm; from the second layer to the third layer; or from the second layer to the last of the additional layers; wherein the ceramic multilayer blank is substantially, preferably completely, free of any oxide of the element Mn.

In one embodiment, the content of the oxides of the elements Mn and Co in the respective ceramic material of the respective layer of the multilayer blank is continuously increasing from the second layer to the third layer; or from the second layer via the first of the additional layers continuously further to the last of the additional layers.

In one embodiment, the content of the oxides of the elements Mn and Co is continuously increasing for the oxide of the element Mn by totally 1 to 35 ppm, preferably by 1 to 20 ppm, and more preferably by 1 to 6 ppm; while continuously increasing for the oxide of the element Co by totally 1 to 70 ppm, preferably by 1 to 40 ppm, and more preferably by 1 to 20 ppm; from the second layer to the third layer; or from the second layer to the last of the additional layers.

In one embodiment, the content of the oxides of the elements Mn and Co in the respective ceramic material of the respective layer of the multilayer blank is continuously increasing from the second layer to the third layer; or from the second layer via the first of the additional, layers continuously further to the last of the additional layers; and

the content of the oxides of the elements Fe, Tb, Pr and Er in the respective ceramic material of the respective layer of the multilayer blank is continuously decreasing from the second layer to the third layer; or from the second layer via the first of the additional layers continuously further to the last of the additional layers.

In one embodiment, the ceramic material of all layers of the ceramic multilayer blank comprises zirconium dioxide doped with yttrium oxide (Y2O3), calcium oxide (CaO), magnesium oxide (MgO) and/or cerium oxide (CeO2), wherein the zirconium dioxide is doped with yttrium oxide, wherein the percentage of yttrium oxide in the second, third and/or additional layers is between 1 wt % and 15 wt %, preferably between 2 wt % and 11 wt %, and more preferably between 2.5 wt % and 10 wt %.

In one embodiment, the ceramic material of all layers of the ceramic multilayer blank comprises zirconium dioxide doped with yttrium oxide (Y2O3), calcium oxide (CaO), magnesium oxide (MgO) and/or cerium oxide (CeO2), wherein the zirconium dioxide is doped with yttrium oxide, wherein the percentage of yttrium oxide in the pink colored layer is between 0.3 wt % and 10.5 wt %, preferably between 0.6 wt % and 7.5 wt %, and more preferably between 0.75 wt % and 7 wt %.

The method outlined above defines that after subsequently filling of a first and a second layer, the second layer is provided with a surface structure such that when viewed along its surface comprises regions of different heights, i.e., it does not have a uniform fill height, and a third layer or a first additional layer that differs from the second layer in its composition is then filled into the mold.

As an alternative, an intermediate layer with a different composition compared to the second layer is filled into the mold on top of the second layer and both layers are mixed before the third layer or the first additional layer is then filled into the die. It is in particular provided for the material of the intermediate layer to be mixed with the material of the third layer or the first additional layer starting from the free surface of the intermediate layer over a height, which is twice or approximately twice the height of the intermediate layer. Furthermore, it is in particular provided for the material of the intermediate layer to be the same material as that used for the third layer or the first additional layer.

In accordance with the invention, for the first alternative initially a first and a second layer of material in pourable condition is filled into a mold. The material of the second layer may be a tooth-colored zirconium dioxide granular material that, for example, has a bulk density between 1 g/cm³ and 1.4 g/cm³, preferably in the range between 1.15 g/cm³ and 1.35 g/cm³. After filling of the granular material of the second layer, which can have a grain size D50 between 40 μm and 70 μm, the surface is smoothed, and then to shape or form a structure that has depressions (valleys) and elevations which in particular extend parallel to one another, in particular however, concentric or parallel to one another. For this purpose it is in particular provided that the structure is formed through an element that moves relative to the second layer, in particular rotates relative to the second layer, that in particular with a wave-like, comb-like or saw-tooth-like section structures the second layer in its surface region. There is a quasi “raking” of the second layer surface to form the structure with alternating elevations and valleys.

In particular the structure is to be formed such that the volume of the elevations is equal to, or approximately equal to, that of the depressions or valleys.

The saw-tooth-like element preferably has V-shaped teeth that are symmetrical in shape and has flanks that enclose an angle between 15° and 45°. The distance between neighbouring teeth, i.e., the distance from peak to peak, should be between 1 mm and 4 mm, preferably between 1 mm and 3 mm.

The pourable third or first additional ceramic material is then filled into the mold, and increases in quantity starting from the troughs that form the valleys of the structure, so that as a consequence, there is a virtually constant increase in the percentage of the third layer or the first additional layer across the height, of the elevations. After smoothing of the surface, the layers are compressed to achieve an approximate density in the region of 3 g/cm³. Pre-sintering is then carried out at a temperature between 700° C. and 1100° C., in particular in the range between 800° C. and 1000° C. for a time between, for example, 100 and 150 minutes. The blank so produced is then worked, for example, through milling and/or grinding to yield a desired dental restoration that is then sintered until a final density can be attained that for zirconium dioxide, for example, is in the range 6.0 to 6.1 g/cm³.

Complete/final sintering to full density is carried out, for example, for a time between 10 minutes and 250 minutes at a temperature in the range 1300° C. to 1600° C. Complete sintering may also be carried out at a somewhat higher temperature. If sintering is carried out at a temperature that is, for example, 100° C. higher than that given by the manufacturer of the starting material, then this is referred to as over-sintering, with a sintering time corresponding to that given for complete sintering.

Complete sintering is in particular carried out in the range 1350° C. to 1550° C., wherein densities between 6.0 and 6.1 g/cm³, in particular between 6.04 and 6.09 g/cm³ can be achieved.

The penetration of the second and, the third or the additional, layers results in the advantage that different physical and optical properties can be achieved across the height of the blank. Thus, once the second layer is colored to the required extent, a tooth-colored edge region can be obtained after complete sintering, across the transition region formed by the penetrating second and, third layer or first additional layer, materials, in which the intensity (a synonym to this expression “intensity” in the sense of the present invention is the expression “chroma”) of the tooth color decreases continuously and at the same time the translucency increases in the desired manner. The dental restoration is then produced from the blank, in particular by milling, taking into account the course of the layer, wherein the dental restoration is “laid” in the blank such that the tooth incisor extends in the region of the third layer or the first additional layer.

Independently thereof, a continuous transition between the second and, the third or the additional, layers is provided on the basis of the teaching of the invention, so that color/translucency decreases or increases continuously and also the bending strength can be adjusted in such a way that the region of the dental restoration, which is subject to an extensive loading, has a higher bending strength than the regions which are not so heavily loaded.

In a preferred manner, the possibility of mixing the second and, the third or the additional, layer materials is provided by rotating an element, in particular, about an axis extending along the longitudinal axis of the mold, in order to achieve the structure, which is also referred to as a wave-like or saw-tooth-like structure, by displacing material of the surface of the layer. There is also the possibility of forming the structure by means of a pressure element which acts on the second layer in the direction of the surface and which has, in particular, elevations extending in its surface with depressions extending between them so that the negative form of the element, also referred to as a stamp, is impressed into the surface of the second layer. Then, as explained above, the ceramic material of the third layer or the first additional layer is filled and then smoothed to press the layers together and then pre-sinter the pressed object.

The invention is also characterized in that the second and, the third layer or the first additional layer, are mutually penetrated in their superposed regions across a height H which is a 1/15 to a quarter, in particular 1/10 to ⅕, of the total height of the second and, third layer or first additional, layers.

The second layer should have a height in an unstructured state which corresponds approximately to ½ to ⅔ of the sum of the second and, third layer or first additional, layers.

The invention is characterized, inter alia, by the following measures. First, a first layer of a first ceramic material and a second layer of a second ceramic material are filled in pourable condition layer-by-layer into a mold. Herein, the first layer and the second layer are made of ceramic materials of different compositions; wherein the first layer is a pink colored layer, wherein the first ceramic material comprises 2 to 25 wt %, preferably 4 to 17 wt %, and more preferably 5 to 12 wt %, erbium oxide; and wherein the second layer is a first tooth-colored layer, wherein the ceramic material of the second layer consists predominantly of zirconium dioxide.

The filling height of the second layer corresponds approximately to ½ to ⅔ blank height of second and, third or all additional, layers before pressing.

The surface is then structured by a specially structured element or a stamp, wherein the structure can be designed such that there is a continuous transition of the properties from the second material to the second and, third layer or first additional, material. Also, the surface geometry of the second layer can be aligned with the diffusion coefficients of the layer materials.

Preferably, a rotating element is used which is lowered into the mold, i.e., into the mold, in which the second layer is located, and then is immersed into the second layer to the extent required. The surface is selectively structured by rotating the element, which is structured on the layer side like a wave-like or comb-like element. Alternatively, the surface may be structured by a press plunger with a suitable geometry.

Subsequently, the mold is filled with the second and the, third or first additional, ceramic material. The usual pressing of the ceramic materials and pre-sintering then takes place.

There is also no departure from the invention if a material for forming an intermediate layer is then filled into the mold after the filling of the second layer.

The intermediate layer may, for example, have a height of 1/10 to ⅕ of the total height of the second and, the third or the additional, layers to be filled into the mold. The intermediate layer material is then mixed with the second layer. In this case, mixing takes place with an element which at least penetrates into the second layer to a depth which corresponds to the height of the intermediate layer. Then, a layer corresponding to the previously described third or first additional layer is filled into the mold. As explained above, the ceramic materials are then pressed to a blank and pre-sintered to obtain, in particular, a dental restoration from the blank so produced by milling. A further processing step is complete sintering to full density. The material of the intermediate layer should be that of the third or first additional layer.

The above method of the present invention has been found suitable for the production of a full or a partial denture, an implant supported superstructure, or an implant supported denture.

In the sense of the present invention, the inventive method can therefore (as described above) be applied to provide a multilayer blank, which is presintered or full sintered at the end of the inventive method.

The inventive method provides thereby a ceramic multilayer blank, which can be used by a skilled person to digitally position a required dental restoration as listed above in the blank, wherein it has to be paid attention to position the future area of the human gingiva completely inside of the first ceramic layer of the blank while the future dentin area shall be positioned entirely in the other layers (second and third ceramic layer; or second and additional layers) of the blank. After having produced the dental restoration out of the blank, the skilled person has still to subsequent work in the transition area of the human gingiva to the dentin area by applying stains or glaze materials before finally glazing the dental restoration.

The present invention thus addresses the problem of provide a method to produce a pink colored ceramic material for dental restoration applications wherein said pink colored ceramic material shall be as similar as possible to human gingiva.

The following non-limiting examples are provided to illustrate an embodiment of the present invention and to facilitate understanding of the invention but are not intended to limit the scope of the invention, which is defined by the claims appended hereto.

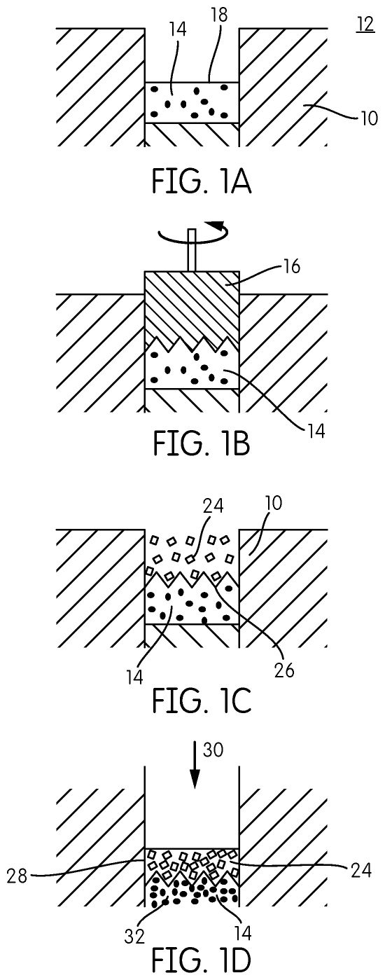

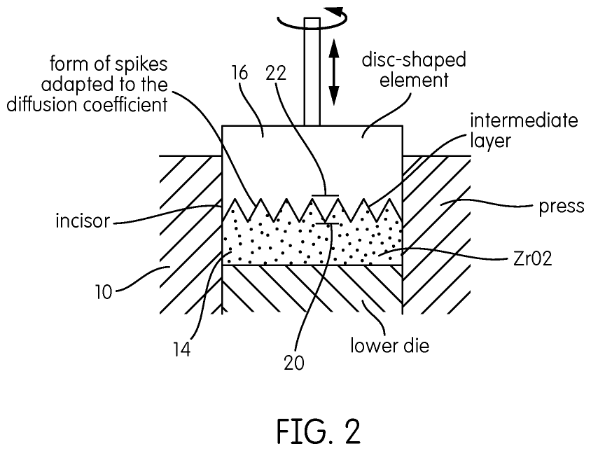

With reference to FIGS. 1 and 2, the production of a blank from which a corresponding dental restoration can be produced is described.

The provision of a first layer of a first ceramic material is not explicitly illustrated in all Figures even when foreseen in all embodiments of the present invention. Thus, as described before a first layer of a first ceramic material is always filled first in pourable condition into a mold, wherein the first ceramic material comprises 2 to 25 wt %, preferably 4 to 17 wt %, and more preferably 5 to 12 wt %, erbium oxide.

Then, as shown in FIG. 1a) a second material 14 is filled subsequently on the top of the surface of the first layer of the first ceramic material into the mold (other synonym for the expression “mold” is the expression “die”) 10 of a press 12.

Subsequently, a third or first additional layer 24 is filled into the die 10 (FIG. 1c), wherein the total height of the layers 14 and 24 is equal to twice the height of the layer 14 in the unstructured state without restriction of the teaching according to the invention.

If the second layer 14 preferably has a height which corresponds to half the total height H of the second and, the third or first additional layer, 14, 24, then the height of the second layer 14 can also be ½ H to ⅔ H and thus that of the third or first additional layer 24 ⅓ H to ½ H.

The smoothed surface is then structured according to step b). For this purpose, for example, a disc-shaped or plate-shaped or web-shaped element 16 is used, which in the example embodiment has a toothed geometry on the layer side, so that a corresponding negative structure is formed in the surface 18 of the layer 14 by displacing material. This structure is represented by concentrically extending elevations and surrounding valleys. The distance between the elevation (peak) and the valley (depression), i.e., the clear distance between the projection 20 and the valley bottom 22 according to FIG. 2, should be approximately ⅕ of the height of all layers.

In particular, it is provided that the structure is formed such that the volume of the elevations is equal to or approximately equal to the volume of the depressions or valleys.

Since the material of the third or first additional layer 24 penetrates to the base of the valleys 26 in the surface 18 of the layer 14, there is a continuous transition between the properties of the layer 14 and the layer 24, after the layers 24, 14 have been pressed according to FIG. 1d). The transition or intermediate layer is denoted by the reference numeral 28 in FIG. 1d).

The layers 14, 24 are pressed by means of a stamp 30, with a pressure between 1000 bar and 8880 bar.

The pourable material, i.e., in the state in which it is filled into the die 10, has a bulk density between 1 g/cm³ and 1.4 g/cm³. After pressing, the density is approximately between 3.0 and 3.5 g/cm³.

As a result of the structuring, a density of up to 2 g/cm³ is obtained in the transition region between the unmixed regions of the second and the, third or first additional, layers 14, 24 before the layers 14 and 24 are compacted. The transition region can also be referred to as middle layer 28.

After pressing, the produced blank is ejected from the mold 10 and pre-sintered in the customary manner at a temperature of between 800° C. and 1000° C. for a period of time between 100 minutes and 150 minutes.

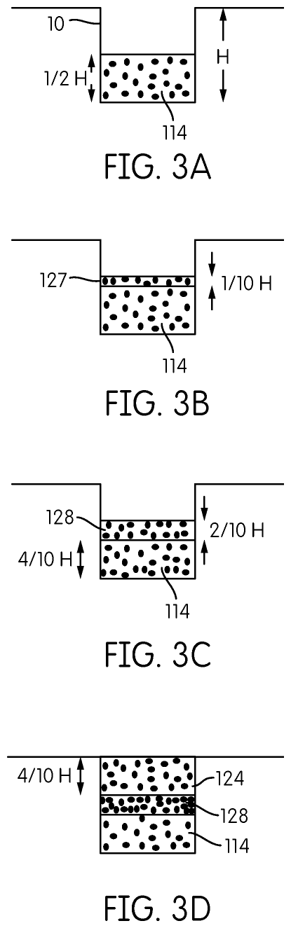

With reference to FIG. 3 an alternative method, which follows the teaching according to the invention, wherein a blank or a dental restoration is to be prepared which provides a largely continuous transition between a second layer and a third or first additional layer.

Also herein, a first layer of a first ceramic material is filled first in pourable condition into a mold, wherein the first ceramic material comprises 2 to 25 wt %, preferably 4 to 17 wt %, and more preferably 5 to 12 wt %, erbium oxide.

Then, according to FIG. 3a, a second ceramic material, which corresponds to the layer 14 according to FIG. 1, is filled subsequently into a die 10. The corresponding layer in FIG. 3a is indicated by the numeral 114. The height of this layer may be half the height of the total layers which are filled into the die 10. A layer 127 with a thickness which in the example embodiment is 1/10 of the total height of the layers is then applied to the layer 114. The material of the layer 127 can correspond to that of the third or first additional layer 24 according to FIG. 1. The layer 127 is then mixed with a surface region of the layer 114 over a depth corresponding to the thickness of the layer 127. This forms an intermediate layer 128 having a thickness of 2/10 of the total height of the layers. A further layer 124, which corresponds to the third or first additional layer 24 according to FIG. 1, is then applied to the intermediate layer 128. The height of the layer 124 in the example embodiment is thus 4/10 of the total height H. The layers 124, 128, 114 are then pressed together in accordance with the example embodiment of FIG. 1 to enable performance of the process steps pre-sintering, working and complete sintering as described. Working can naturally be carried out after complete sintering.

It can be seen that the invention can also be described with reference to one or more of the following combinations:

A. A method for producing a ceramic multilayer blank comprising at least a first layer of a first ceramic material and at least a second layer of a second ceramic material, wherein the first layer and the second layer are made of ceramic materials of different compositions, which are filled in pourable condition layer-by-layer into a mold and thereafter they are pressed and then sintered, characterized in that the first layer is a pink colored layer, wherein the first ceramic material comprises 2 to 25 wt %, preferably 4 to 17 wt %, and more preferably 5 to 12 wt %, erbium oxide.

B. Method according to claim 1, characterized in that the ceramic multilayer blank additionally comprises at least a third layer, wherein said third layer is made of a third ceramic material, which has a different composition than the first layer, and which has a different or identical composition than the second layer; wherein after filling of the second layer of the second ceramic material in pourable condition, surface of the second layer is structured in such a way that the second layer when viewed across its surface differs from region to region in its height, and then as the third layer, a layer of a third ceramic material in pourable condition is filled into the mold; or wherein after filling of the second layer of the second ceramic material in pourable condition, an intermediate layer of an intermediate ceramic material in pourable condition, which differs from the second layer, is filled into the mold; wherein the second ceramic material of the second layer is mixed with the third ceramic material of the third layer to form said intermediate layer of an intermediate ceramic material; and then the third layer of a third ceramic material in pourable condition is filled into the mold.

C. Method according to claim 1, characterized in that the ceramic multilayer blank further comprises a plurality of additional layers, wherein each of said additional layers is made of a ceramic material, which has a different composition than the first layer, and which has a different or identical composition than the second layer; wherein after filling of the second layer of the second ceramic material in pourable condition, surface of the second layer is structured in such a way that the second layer when viewed across its surface differs from region to region in its height, and then as the first of said additional layers, a layer of a ceramic material of said additional layers in pourable condition is filled into the mold; wherein after filling of the first of said additional layers of a ceramic material of said additional layers in pourable condition, surface of the first of said additional layers is structured in such a way that the first of said additional layers when viewed across its surface differs from region to region in its height, and then as the second of said additional layers, a layer of a ceramic material of said additional layers in pourable condition is filled into the mold; wherein if the second of said additional layers is not the last additional layer, the aforementioned method step is repeated until all additional layers in pourable condition are filled into the mold.

D. Method according to claim 2 or 3, characterized in that the surface of the respective layer is structured in such a way that elevations and depressions are provided.

E. Method according to claim 1, characterized in that the ceramic multilayer blank further comprises a plurality of additional layers, wherein each of said additional layers is made of a ceramic material, which has a different composition than the first layer, and which has a different or identical composition than the second layer; wherein after filling of the second layer of the second ceramic material in pourable condition, a first intermediate layer of a first intermediate ceramic material in pourable condition, which differs from the second layer, is filled into the mold; wherein the second ceramic material of the second layer is mixed with the ceramic material of the first of said additional layers to form said first intermediate layer of a first intermediate ceramic material; and then the first of said additional layers of a ceramic material in pourable condition is filled into the mold; wherein after filling of the first of said additional layers of a ceramic material in pourable condition, a second of said additional layers of a ceramic material of said additional layers in pourable condition is filled into the mold; wherein the ceramic material of the first of said additional layers is mixed with the ceramic material of the second of said additional layers to form said second intermediate layer of a second intermediate ceramic material, and then the second of said additional layers is filled into the mold; wherein if the second of said additional layers is not the last additional layer, the aforementioned method step is repeated until all additional layers in pourable condition are filled into the mold.

F. Method according to claim 3, 4 or 5, characterized in that the ceramic multilayer blank comprises two to five, preferably two to four, more preferably three, additional layers, wherein each of said additional layers is made of a ceramic material, which has a different composition than the first layer, and which has a different or identical composition than the second layer.

Method according to claim 2, characterized in that the ceramic material of the second layer and/or the ceramic material of the third layer comprises at least one oxide of the elements Mn, Co, Fe, Tb, Pr and Er; and/or wherein the ceramic material of the first layer further comprises at least one oxide of the elements Mn, Co, Fe, Tb and Pr.

H. Method according to claim 3, characterized in that the ceramic material of the second layer and/or the ceramic material of all additional layers comprises at least one oxide of the elements Mn, Co, Fe, Tb, Pr and Er; and/or wherein the ceramic material of the first layer further comprises at least one oxide of the elements Mn, Co, Fe, Tb and Pr.

I. Method according to claim 7 or 8, characterized in that the ceramic material of the first layer and/or the ceramic material of the second layer and/or the ceramic material of the third layer and/or the ceramic material of all additional layers of the ceramic multilayer blank further comprises between 0.0005 and 0.02 wt %, preferably between 0.0005 and 0.01 wt %, and more preferably between 0.0005 and 0.05 wt % of an oxide of the element Mn; and/or

the ceramic material of the first layer and/or the ceramic material of the second layer and/or the ceramic material of the third layer and/or the ceramic material of all additional layers of the ceramic multilayer blank further comprises between 0.0005 and 0.1 wt %, preferably between 0.0005 and 0.07 wt %, and more preferably between 0.0005 and 0.05 wt % of an oxide of the element Co.

J. Method according to claim 9, characterized in that the content of the at least one oxide of the element. Mn in the respective ceramic material of the respective layer of the multilayer blank is continuously increasing from the second layer to the third layer; or from the second layer via the first of the additional layers continuously further to the last of the additional layers.

K. Method according to claim 10, characterized in that the content of the at least one oxide of the element Mn is continuously increasing by totally 1 to 50 ppm, preferably by 1 to 30 ppm, and more preferably by 1 to 10 ppm; from the second layer to the third layer; or from the second layer to the last of the additional layers; wherein the ceramic multilayer blank is substantially, preferably completely, free of any oxide of the element Co.

L. Method according to claim 9, characterized in that the content of the at least one oxide of the element Co in the respective ceramic material of the respective layer of the multilayer blank is continuously increasing from the second layer to the third layer; or from the second layer via the first of the additional layers continuously further to the last of the additional layers.

M. Method according to claim 12, characterized in that the content of the at least one oxide of the element Co is continuously increasing by totally 1 to 100 ppm, preferably by 1 to 60 ppm, and more preferably by 1 to 30 ppm; from the second layer to the third layer; or from the second layer to the last of the additional layers; wherein the ceramic multilayer blank is substantially, preferably completely, free of any oxide of the element Mn.

N. Method according to claim 9, characterized in that the content of the oxides of the elements Mn and Co in the respective ceramic material of the respective layer of the multilayer blank is continuously increasing from the second layer to the third layer; or from the second layer via the first of the additional layers continuously further to the last of the additional layers.

O. Method according to claim 14, characterized in that the content of the oxides of the elements Mn and Co is continuously increasing for the oxide of the element Mn by totally 1 to 35 ppm, preferably by 1 to 20 ppm, and more preferably by 1 to 6 ppm; while continuously increasing for the oxide of the element Co by totally 1 to 70 ppm, preferably by 1 to 40 ppm, and more preferably by 1 to 20 ppm; from the second layer to the third layer; or from the second layer to the last of the additional layers.

P. Method according to claim 9, characterized in that the content of the oxides of the elements Mn and Co in the respective ceramic material of the respective layer of the multilayer blank is continuously increasing from the second layer to the third layer; or from the second layer via the first of the additional layers continuously further to the last of the additional layers; and the content of the oxides of the elements Fe, Tb, Pr and Er in the respective ceramic material of the respective layer of the multilayer blank is continuously decreasing from the second layer to the third layer; or from the second layer via the first of the additional layers continuously further to the last of the additional layers.

Q. Method according to one of the preceding claims, characterized in that the ceramic material of all layers of the ceramic multilayer blank comprises zirconium dioxide doped with yttrium oxide (Y2O3), calcium oxide (CaO), magnesium oxide (MgO) and/or cerium oxide (CeO2), wherein the zirconium dioxide is doped with yttrium oxide, wherein the percentage of yttrium oxide in the second, third and/or additional layers is between 1 wt % and 15 wt %, preferably between 2 wt % and 11 wt %, and more preferably between 2.5 wt % and 10 wt %.

R. Method according to one of the preceding claims, characterized in that the ceramic material of all layers of the ceramic multilayer blank comprises zirconium dioxide doped with yttrium oxide (Y2O3), calcium oxide (CaO), magnesium oxide (MgO) and/or cerium oxide (CeO2), wherein the zirconium dioxide is doped with yttrium oxide, wherein the percentage of yttrium oxide in the pink colored layer is between 0.3 wt % and 10.5 wt %, preferably between 0.6 wt % and 7.5 wt %, and more preferably between 0.75 wt % and 7 wt %.

While the principles of the invention have been explained in relation to certain particular embodiments, and are provided for purposes of illustration, it is to be understood that various modifications thereof will become apparent to those skilled in the art upon reading the specification. Therefore, it is to be understood that the invention disclosed herein is intended to cover such modifications as fail within the scope of the appended claims. The scope of the invention is limited only by the scope of the appended claims.

1. A method for producing a ceramic multilayer blank comprising the steps of:
filling at least a first layer of a first ceramic material in pourable condition into a mold; and
filling at least a second layer of a second ceramic material in pourable condition into the mold;
wherein the first layer and the second layer are made of ceramic materials of different compositions, which are filled in pourable condition layer-by-layer into the mold;
pressing the first and second ceramic material within the mold and then sinter the mold,
wherein the first layer is a pink colored layer; and
wherein the first ceramic material includes 2 to 25 wt % erbium oxide.

2. Method according to claim 1, further comprising the step of filling at least a third layer of a third ceramic material in a pourable condition into the mold, wherein the third layer has a different composition than the first layer, and has a different or identical composition than the second layer;
wherein after filling of the second layer of the second ceramic material in pourable condition, a surface of the second layer is structured in such a way that the second layer when viewed across its surface differs from region to region in its height; or
wherein after filling of the second layer of the second ceramic material in pourable condition, filling the mold with an intermediate layer of an intermediate ceramic material in pourable condition, which differs from the second layer; and then filling the mold with a third layer of a third ceramic material in a pourable condition;
wherein the second ceramic material of the second layer is mixed with the third ceramic material of the third layer to form said intermediate layer of an intermediate ceramic material.

3. Method according to claim 1 further comprising the steps of milling the mold with a plurality of additional layers, wherein each of said additional layers is made of a ceramic material, which has a different composition than the first layer, and which has a different or identical composition than the second layer;
wherein after filling of the second layer of the second ceramic material in pourable condition a surface of the second layer is structured in such a way that the second layer when viewed across its surface differs from region to region in its height, and thereafter, a first layer of a ceramic material of said additional layers in pourable condition is filled into the mold;
wherein after filling of the first layer of said additional layers of the ceramic material of said additional layers in pourable condition, a surface of the first layer of said additional layers is structured in such a way that the first layer of said additional layers when viewed across its surface differs from region to region in its height, and then as a second layer of a ceramic material of said additional layers in pourable condition is filled into the mold; and
wherein if the second layer of said additional layers is not the last additional layer, the aforementioned method step is repeated until all additional layers in pourable condition are filled into the mold.

4. Method according to claim 2, wherein the surface of the respective layer is structured in such a way that elevations and depressions are provided.

5. Method according to claim 1, wherein the ceramic multilayer blank further comprises a plurality of additional layers, wherein each of said additional layers is made of a ceramic material, which has a different composition than the first layer, and which has a different or identical composition than the second layer;
wherein after filling of the second layer of the second ceramic material in pourable condition, a first intermediate layer of a first intermediate ceramic material in pourable condition, which differs from the second layer, is filled into the mold; wherein the second ceramic material of the second layer is mixed with the ceramic material of the first of said additional layers to form said first intermediate layer of a first intermediate ceramic material; and then the first of said additional layers of a ceramic material in pourable condition is filled into the mold;
wherein after filling of the first of said additional layers of a ceramic material in pourable condition, a second of said additional layers of a ceramic material of said additional layers in pourable condition is filled into the mold; wherein the ceramic material of the first of said additional layers is mixed with the ceramic material of the second of said additional layers to form said second intermediate layer of a second intermediate ceramic material, and then the second of said additional layers is filled into the mold;
wherein if the second of said additional layers is not the last additional layer, the aforementioned method step is repeated until all additional layers in pourable condition are filled into the mold.

6. Method according to claim 3, wherein the ceramic multilayer blank includes two to five additional layers, wherein each of said additional layers is made of a ceramic material, which has a different composition than the first layer, and which has a different or identical composition than the second layer.

7. Method according to claim 2, wherein the ceramic material of the second layer and/or the ceramic material of the third layer includes at least one oxide of the elements selected from the group consisting of Mn, Co, Fe, Tb, Pr and Er; and wherein the ceramic material of the first layer further comprises at least one oxide of the elements selected from the group consisting of Mn, Co, Fe, Tb and Pr.

8. Method according to claim 3, wherein the ceramic material of the second layer and/or the ceramic material of all additional layers comprises at least one oxide of the elements selected from the group consisting of Mn, Co, Fe, Tb, Pr and Er; and/or wherein the ceramic material of the first layer further comprises at least one oxide of the elements selected from the group consisting of Mn, Co, Fe, Tb and Pr.

9. Method according to claim 7, wherein the ceramic material of the first layer and/or the ceramic material of the second layer and/or the ceramic material of the third layer and/or the ceramic material of all additional layers of the ceramic multilayer blank further comprises between 0.0005 and 0.02 wt % of an oxide of the element Mn; and
the ceramic material of the first layer and/or the ceramic material of the second layer and/or the ceramic material of the third layer and/or the ceramic material of all additional layers of the ceramic multilayer blank further comprises between 0.0005 and 0.1 wt % of an oxide of the element Co.

10. Method according to claim 9, wherein the content of the at least one oxide of the element Mn in the respective ceramic material of the respective layer of the multilayer blank is continuously increasing from the second layer to the third layer; or from the second layer via the first of the additional layers continuously further to the last of the additional layers.

11. Method according to claim 10, wherein the content of the at least one oxide of the element Mn is continuously increasing by totally 1 to 50 ppm from the second layer to the third layer; or from the second layer to the last of the additional layers; wherein the ceramic multilayer blank is substantially or completely, free of any oxide of the element Co.

12. Method according to claim 9, wherein the content of the at least one oxide of the element Co in the respective ceramic material of the respective layer of the multilayer blank is continuously increasing from the second layer to the third layer; or from the second layer via the first of the additional layers continuously further to the last of the additional layers.

13. Method according to claim 12, wherein the content of the at least one oxide of the element Co is continuously increasing by totally 1 to 100 ppm from the second layer to the third layer; or from the second layer to the last of the additional layers; wherein the ceramic multilayer blank is substantially or completely free of any oxide of the element Mn.

14. Method according to claim 9, wherein the content of the oxides of the elements Mn and Co in the respective ceramic material of the respective layer of the multilayer blank is continuously increasing from the second layer to the third layer; or from the second layer via the first of the additional layers continuously further to the last of the additional layers.

15. Method according to claim 14, wherein the content of the oxides of the elements Mn and Co is continuously increasing for the oxide of the element Mn by totally 1 to 35 ppm while continuously increasing for the oxide of the element Co by totally 1 to 70 ppm from the second layer to the third layer; or from the second layer to the last of the additional layers.

16. Method according to claim 9, wherein the content of the oxides of the elements Mn and Co in the respective ceramic material of the respective layer of the multilayer blank is continuously increasing from the second layer to the third layer; or from the second layer via the first of the additional layers continuously further to the last of the additional layers; and
the content of the oxides of the elements Fe, Tb, Pr and Er in the respective ceramic material of the respective layer of the multilayer blank is continuously decreasing from the second layer to the third layer; or from the second layer via the first of the additional layers continuously further to the last of the additional layers.

17. Method according to claim 1, wherein the ceramic material of all layers of the ceramic multilayer blank comprises zirconium dioxide doped with yttrium oxide (Y2O3), calcium oxide (CaO), magnesium oxide (MgO) and/or cerium oxide (CeO2), wherein the zirconium dioxide is doped with yttrium oxide, wherein the percentage of yttrium oxide in the second, third and/or additional layers is between 1 wt % and 15 wt %.

18. Method according to claim 1, wherein the ceramic material of all layers of the ceramic multilayer blank comprises zirconium dioxide doped with yttrium oxide (Y2O3), calcium oxide (CaO), magnesium oxide (MgO) and/or cerium oxide (CeO2), wherein the zirconium dioxide is doped with yttrium oxide, wherein the percentage of yttrium oxide in the pink colored layer is between 0.3 wt % and 10.5 wt %.
